SEQUENCE LISTING (20 sequences)

SEQ ID NO: 1 - DNA, 405 bp, Homo sapiens
ttattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcatatcattggatgggactagtacacattccaacaaatggatcttggcagtgggaagatggctccattctctcacccaacctactaacaataattgaaatgcagaagggagactgtgcactctatgcctcgagctttaaaggctatatagaaaactgttcaactccaaatacatacatctgcatgcaaaggactgtg

SEQ ID NO: 2 - protein, 216 aa, Homo sapiens
MGWIRGRRSRHSWEMSEFHNYNLDLKKSDFSTRWQKQRCPVVKSKCRENASPFFFCCFIAVAMGIRFIIMVTIWSAVFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 3 - DNA, 282 bp, Homo sapiens
atgatccatctgggtcacatcctcttcctgcttttgctcccagtggctgcagctcagacgactccaggagagagatcatcactccctgccttttaccctggcacttcaggctcttgttccggatgtgggtccctctctctgccgctcctggcaggcctcgtggctgctgatgcggtggcatcgctgctcatcgtgggggcggtgttcctgtgcgcacgcccacgccgcagccccgcccaagaagatggcaaagtctacatcaacatgccaggcaggggctga

SEQ ID NO: 4 - protein, 93 aa, Homo sapiens
MIHLGHILFLLLLPVAAAQTTPGERSSLPAFYPGTSGSCSGCGSLSLPLLAGLVAADAVASLLIVGAVFLCARPRRSPAQEDGKVYINMPGRG

SEQ ID NO: 5 - DNA, 693 bp, Homo sapiens
cccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaa

SEQ ID NO: 6 - protein, 231 aa, Homo sapiens
PKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - DNA, 72 bp, Homo sapiens
atctacatctgggcgcccctggccgggacttgtggggtccttctcctgtcactggttatcaccctttactgc

SEQ ID NO: 8 - protein, 24 aa, Homo sapiens
IYIWAPLAGTCGVLLLSLVITLYC

SEQ ID NO: 9 - DNA, 126 bp, Homo sapiens
gtgaggagtaagaggagcaggctcctgcacagtgactacatgaacatgactccccgccgccccgggcccacccgcaagcattaccagccctatgccccaccacgcgacttcgcagcctatcgctcc

SEQ ID NO: 10 - protein, 42 aa, Homo sapiens
VRSKRSRLLHSDYMNMTPRRPGPTRKHYQPYAPPRDFAAYRS

SEQ ID NO: 11 - DNA, 126 bp, Homo sapiens
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 12 - protein, 42 aa, Homo sapiens
KRGRKKLLYIFKQPFMRPVQTTQEEDGCSCRFPEEEEGGCEL

SEQ ID NO: 13 - DNA, 336 bp, Homo sapiens
agagtgaagttcagcaggagcgcagagccccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 14 - protein, 112 aa, Homo sapiens
RVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - DNA, 212 bp, Artificial Sequence
  synthesized
gggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacag

SEQ ID NO: 16 - DNA, 18 bp, Artificial Sequence
  synthesized
gaaggagaggccaccatg

SEQ ID NO: 17 - DNA, 51 bp, Homo sapiens
atgccgctgctgctactgctgcccctgctgtgggcaggggccctggctatg

SEQ ID NO: 18 - protein, 17 aa, Homo sapiens
MPLLLLLPLLWAGALAM

SEQ ID NO: 19 - DNA, 578 bp, Artificial Sequence
  synthesized
ctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacacat

SEQ ID NO: 20 - DNA, 591 bp, Artificial Sequence
  synthesized
gtgaggagtaagaggagcaggctcctgcacagtgactacatgaacatgactccccgccgccccgggcccacccgcaagcattaccagccctatgccccaccacgcgacttcgcagcctatcgctccaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagagccccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

(19) United States
(12) Patent Application Publication
(10) Pub. No.: US 2021/0309713 A1
(43) Pub. Date: Oct. 7, 2021

(54) Chimeric Antigen Receptor and Method for Treating Cancers

(71) Applicant: DAREN BIOTECH LIMITED, Hong Kong (CN)

(72) Inventor: Yong XIE, Hong Kong (CN)

(73) Assignee: DAREN BIOTECH LIMITED, Hong Kong (CN)

(21) Appl. No.: 15/734,983

(22) PCT Filed: Apr. 3, 2019
(86) PCT No.: PCT/CN2019/081286

§ 371 Date: Dec. 3, 2020

(30) Foreign Application Priority Data
Apr. 4, 2018 (CN) 201810299324.8

(51) Int. Cl. C07K 14/705; C07K 16/28; C12N 15/86; C07K 14/725; A61P 35/00; A61K 35/17

(52) U.S. Cl. CPC C07K 14/70521; C07K 16/2851; C12N 15/86; C07K 14/7051; A61K 38/00; C07K 14/70578; A61P 35/00; A61K 35/17; C07K 14/70517

(57) ABSTRACT

The present disclosure provides a chimeric antigen receptor and a combination of the chimeric antigen receptor and DAP10. The present disclosure provides an expression vector and a host cell for expressing the chimeric antigen receptor and said combination of the chimeric antigen receptor and DAP10. The present disclosure also provides use of the chimeric antigen receptor and the combination of the chimeric antigen receptor and DAP10 in the treatment of cancers or in the preparation of pharmaceutical compositions for treating cancers. The chimeric antigen receptor and the drugs provided in the present disclosure can effectively treat liver cancer, lung cancer and the like.

### CROSS-REFERENCE TO RELATED APPLICATIONS

The present application is a 35 U.S.C. 371 national stage filing of International Application No. PCT/CN2019/081286 filed on Apr. 3, 2019, which claims priority to Chinese Patent Application No. 201810299324.8, with a translated title “Chimeric Antigen Receptor and Method for Treating Cancers” filed on Apr. 4, 2018, the disclosure of which is hereby incorporated into the present application by reference in its entirety.

### REFERENCE TO SEQUENCE LISTING SUBMITTED ELECTRONICALLY

The content of the electronically submitted sequence listing, file name Seq-listing-ST25.txt, size 13,515 bytes, and date of creation Jun. 15, 2021, filed herewith, is incorporated herein by reference in its entirety.

### FIELD OF THE INVENTIONS

The present disclosure relates to the field of immunology and pharmacy field. In particular, the disclosure provides a new chimeric antigen receptor and a combination thereof with DAP10. The disclosure also provides a method of using said antigen for treating depression, and pharmaceutical compositions for treating depression and a manufacturing method thereof.

### BACKGROUND

The natural-killer group 2 member D (NKG2D) receptor is a type II transmembrane protein expressed by all nature killer cells, nature killer T cells and γδ+ T cells. In humans, the NKG2D receptor binds with two groups of ligands, UL16-binding proteins (ULBPs: ULBP1, ULBP2, ULBP3, ULBP4, ULBP5, ULBP6) and MHC class I-chain-related proteins AB (MICA/MICB). Expression of human NKG2D ligands is rarely detectable in healthy tissue but is upregulated in tumor cells, during cell stress, and viral infection. Binding of NKG2D and its ligands serves as an activating signal in the immune response against tumor and viral infection.

In recent years, genetically modified chimeric antigen receptor (CAR) T cells were successfully used in autologous therapy in B-cell lymphoma and leukemia. This method was further developed and applied against solid tumors. CAR-modified T cells utilize the target specific property of monoclonal antibody targeting tumor surface antigens in an HLA-independent manner. Upon activation, CAR-T cells show great cytolytic activity against tumor cells, proliferation ability, and homing properties, but do not respond to checkpoint inhibition. However, due to its ability to directly kill the target antigen expressing cells, CAR-T cells are highly toxic to antigen-positive normal cells or tissues, which makes it necessary to construct CARs through highly tumor-specific structures.

The NKG2D receptor-NKG2D ligands system was used for the first time in CAR therapy in 2005. In natural killer cells and T cells, DNAX-activating protein 10 (DAP10) is a cell surface adaptor for NKG2D receptors.

Lung cancer and liver cancer are two leading causes of cancer-related death worldwide. As typical for all solid tumors, multiple hurdles exist in lung cancer and liver cancer therapies. Recurrences of cancer often appears after therapy over solid tumors.

Therefore, safer and more effective CAR-T cells are needed in the CAR-T field against different types of tumors.

### SUMMARY OF THE INVENTIONS

The present disclosure provides a new chimeric antigen receptor (CAR), which comprises: (a) an antigen binding domain including NKG2D or a functional fragment thereof; (b) a transmembrane domain; and (c) an intracellular signaling domain. The present disclosure also provides a new combination of a chimeric antigen receptor with its accessory protein DAP10 or a functional fragment thereof. The present disclosure also provides nucleic acids and vectors encoding said chimeric antigen receptor with/without DAP10, as well as cells expressing said chimeric antigen receptor with/without DAP10. The present disclosure also provides methods for treating cancers or uses for manufacturing medicaments for treating cancers with the chimeric antigen receptor with/without DAP10 of the present disclosure.

The present disclosure provides a chimeric antigen receptor (CAR), which comprises: (a) an antigen binding domain including NKG2D or a functional fragment thereof; (b) a transmembrane domain; and (c) an intracellular signaling domain.

The present disclosure provides a combination of a chimeric antigen receptor (CAR) and an accessory protein, wherein said CAR comprises: (a) an antigen binding domain including NKG2D or a functional fragment thereof; (b) a transmembrane domain; and (c) an intracellular signaling domain, and said accessory protein comprises DAP10 or a functional fragment thereof. In one aspect of the disclosure, said DAP10 has an amino acid sequence of SEQ ID NO: 4.

As used herein, the term “NKG2D”, also known as “NKG2-D”, “CD314”, “KLRK1”, “killer-cell lectin-like receptor subfamily K, member 1”, refers to the killer cell lectin-like receptor subfamily K, member 1 (KLRK1, such as NCBI RefSeq NM_007360) of mammals, especially humans or gene products thereof (such as NCBI RefSeq NP_031386) or naturally occurring variants thereof. In human NK cells and T cells, the ligand-bound form of the NKG2D receptor is a homodimer (Li et al., Nat Immunol 2001; 2:443-451). NKG2D activity includes cell activation, antibody recognition, etc., as well as binding to two kinds of ligands, which are UL16-binding protein (ULBP) and MHC type I chain-associated protein A/B, respectively.

DAP10 (membrane protein 10) is a cell surface adaptor protein, which can form an activating receptor complex with NKG2D, particular refer to humans DAP10 (GenBank: AAG29425.1). The activity of DAP10 includes forming a complex with NKG2D (Wu, J. et al., Science 285 (5428), 730-732, 1999).

In certain embodiments of the present disclosure, the antigen binding domain of said chimeric antigen receptor (CAR) comprises a functional fragment of NKG2D. Said functional fragment can be, for example, an NKG2D fragment corresponding to amino acid residues 82-216 of human NKG2D, that is, an NKG2D fragment comprising amino acids No. 82-216 in the amino acid sequence as set forth in SEQ ID NO: 2:

LFNQEVQIPLTESYCGPCPKNWICYKNNCYQFFDESKNWYESQASCMSQN
ASLLKVYSKEDQDLLKLVKSYHWMGLVHIPTNGSWQWEDGSILSPNLLTI
IEMQKGDCALYASSFKGYIENCSTPNTYICMQRTV.

In certain embodiments of the present disclosure, the antigen binding domain of said chimeric antigen receptor (CAR) comprises a leading peptide. A leading peptide assists the expression of a protein and transports the protein through the cell membrane. Leading peptides known in the field can be used in the present disclosure. In the antigen binding domain of CAR of the present disclosure, the leading peptide can be located upstream of NKD2G or its functional fragments. In certain embodiments of the present disclosure, said leading peptide is a leading peptide of CD33, which has an amino acid sequence of SEQ ID NO: 18. A leading peptide can assist the expression of a CAR on the surface of a cell, yet the leading peptide is not necessary for the CAR to function. In the embodiments of the present disclosure, after expression of CAR on the surface of cells, the leading peptide is cleaved from CAR. In certain embodiments of the present disclosure, said CAR does not include a leading peptide.

In certain embodiments of the present disclosure, said chimeric antigen receptor (CAR) comprises a transmembrane domain. In yet one aspect of the present disclosure, transmembrane domains can be used in the present disclosure include transmembrane domain of T cell receptor α, β, or ζ, CD28, CD3ε, CD45, CD4, CD5, CD8, CD9, CD16, CD22, CD33, CD37, CD64, CD80, CD86, CD134, CD137, CD154, KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), CD160, CD19, IL2Rβ, IL2Rγ, IL7Rα, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (CD150, IPO-3), BLAME (SLAMF8), SELPLG (CD162), LTBR, PAG/Cbp, NKp44, NKp30, NKp46, or the like.

In certain embodiments of the present disclosure, said transmembrane domain of the CAR comprises a transmembrane domain of i) CD8 and/or ii) CD28. In yet some other embodiments of the present disclosure, said transmembrane domain of the CAR comprises a transmembrane domain of CD28 which, for example, can have an amino acid sequence of SEQ ID NO: 8.

In certain embodiments of the present disclosure, said chimeric antigen receptor (CAR) comprises an intracellular signaling domain. In yet some other embodiments, the intracellular signaling domains that can be used in the present disclosure can include the intracellular signaling domain of CD2, CD4, CD5, CD8α, CD8β, CD28, CD134, CD137, ICOS or CD154. Embodiments of the intracellular signaling domains which can be used in the present disclosure include peptides having the following sequences: amino acids 236-351 of CD2 (NCBI RefSeq: NP_001758.2), amino acids 421-458 of CD4 (NCBI RefSeq: NP_000607.1), amino acids 402-495 of CD5 (NCBI RefSeq: NP_055022.2), amino acids 207-235 of CD8a (NCBI RefSeq: NP_001759.3), amino acids 196-210 of CD8β (GenBank: AAA35664.1), amino acids 181-220 of CD28 (NCBI RefSeq: NP_006130.1), amino acids 214-255 of CD137 (4-1BB, NCBI RefSeq: NP_001552.2), amino acids 241-277 of CD134 (OX40, NCBI RefSeq: NP_003318.1) and amino acids 166-199 of ICOS (NCBI RefSeq: NP_036224.1), and variants having substantially same function of the peptides mentioned above.

Preferably, the intracellular signaling domain used in the chimeric antigen receptor (CAR) of the disclosure can include one or more of a intracellular signaling of i) CD28, ii) 4-1BB, and/or iii) CD3ζ. In preferred embodiments of the present disclosure, the intracellular signaling used in the CAR of the disclosure includes CD28, 4-1BB, and CD3ζ. In more preferred embodiments of the present disclosure, the intracellular signaling used in the CAR of the disclosure comprises in the order from the N terminal to the C terminal: CD28, 4-1BB, and CD3ζ. Herein, CD28 is a T cell marker important in T cell co-stimulation. 4-1BB, also known as CD3ζ, transmits a potent costimulatory signal to T cells, promoting differentiation and enhancing long-term survival of T lymphocytes. CD3ζ associates with TCRs to produce a signal and contains immunoreceptor tyrosine-based activation motifs (ITAMs). In certain embodiments for the intracellular signaling domain, CD28, 4-1BB, and CD3ζ portions in the CAR respectively have an amino acid sequence of SEQ ID NO: 10, SEQ ID NO: 12, and SEQ ID NO: 14. In certain embodiments of the present disclosure for the intracellular signaling domain, CD28, 4-1BB, and CD3ζ in the CAR respectively have an amino acid sequence of SEQ ID NO: 10, SEQ ID NO: 12, and SEQ ID NO: 14. In certain embodiments of the present disclosure for the intracellular signaling domain, CD28 in the CAR has an amino acid sequence of SEQ ID NO: 10. In one aspect of the present disclosure, the intracellular signaling domain of 4-1BB in the CAR has an amino acid sequence of SEQ ID NO: 12. In certain embodiment of the present disclosure, the intracellular signaling domain of CD3ζ in the CAR has an amino acid sequence of SEQ ID NO: 14.

In a CAR comprising several intracellular signaling domains, an oligo connector or a peptide linker can be placed between each two intracellular signaling domains. Preferably, a connector is a peptide comprising 2-10 amino acids. For example, a linker is a peptide comprising glycine-serine.

In one aspect of the present disclosure, the chimeric antigen receptor (CAR) of the disclosure comprises: (a) an antigen binding domain including an NKG2D fragment corresponds to amino acid residues 82-216 of human NKG2D; (b) a transmembrane domain of CD28; and (c) an intracellular signaling domain which comprises, in the order from the N terminal to the C terminal, CD28, 4-1BB, and CD3ζ.

In certain embodiments of the present disclosure, said chimeric antigen receptor (CAR) includes a hinge region between said (a) antigen binding domain and said (b) transmembrane domain. Hinge regions can be used in the CAR of the present disclosure includes IgG1H, IgG2H, IgG3H or IgG4H, and the like. In one aspect of the present disclosure, the hinge region between said (a) antigen binding domain and said (b) transmembrane domain comprises IgG1H or a fragment thereof or a variant thereof. For example, the hinge region has an amino acid sequence of SEQ ID NO: 6.

Functional variants of the CARs or the functional fractions thereof (including the antigen binding domain, transmembrane domain, intracellular signaling domain, hinge region and leading peptide) are included in the scope of the disclosure. The term “functional variant” as used herein refers to a CAR, polypeptide, or protein having substantial or significant sequence identity or similarity to a parent CAR, which functional variant retains the biological activity of the CAR of which it is a variant. Functional variants encompass, for example, those variants of the CAR described herein (the parent CAR) that retain the ability to recognize target cells to a similar extent, the same extent, or to a higher extent, as the parent CAR. In reference to the parent CAR, the functional variant can, for instance, be at least about 30%, about 50%, about 75%, about 80%, about 90%, about 98%), about 99% or more identical in amino acid sequence to the parent CAR.

A functional variant can, for example, comprise the amino acid sequence of the parent CAR with at least one conservative amino acid substitution. Alternatively or additionally, the functional variants can comprise the amino acid sequence of the parent CAR with at least one non-conservative amino acid substitution. In this case, it is preferable for the non-conservative amino acid substitution to not interfere with or inhibit the biological activity of the functional variant. The non-conservative amino acid substitution may enhance the biological activity of the functional variant, such that the biological activity of the functional variant is increased as compared to the parent CAR.

The present disclosure also provided an isolated nucleic acid comprising a nucleotide sequence encoding any of the CARs described above. The nucleic acids of the disclosure may comprise a nucleotide sequence encoding any of the leader sequences, antigen binding domains, transmembrane domains, and/or intracellular T cell signaling domains described herein.

In one aspect of the present disclosure, the present disclosure provides an isolated nucleic acid comprising a nucleotide sequence encoding any of the CARs described above, which comprises: (a) an antigen binding domain including NKG2D or a functional fragment thereof (b) a transmembrane domain, and (c) an intracellular signaling domain.

In certain embodiments of the present disclosure, the nucleotide sequence encoding antigen binding domain comprises a nucleic acid sequence encoding an NKG2D functional fragment, preferably an NKG2D fragment corresponds to amino acid residues 82-216 of human NKG2D, for example, the nucleic acid sequence encoding an NKG2D functional fragment comprises a sequence of SEQ ID NO: 1.

In certain embodiments of the present disclosure, the nucleotide sequence encoding the transmembrane domain comprises the nucleotide sequence encoding a transmembrane domain of CD8 and/or CD28. In yet some other embodiments of the present disclosure, the nucleotide sequence encoding the transmembrane domain comprises the nucleotide sequence encoding a transmembrane domain of CD28, for example, the nucleotide sequence comprises a sequence of SEQ ID NO: 7.

In certain embodiments of the present disclosure, the nucleotide sequence encoding the intracellular signaling domain comprises one or more of the nucleotide sequence encoding an intracellular signaling domain of CD28, 4-1BB or CD3ζ. In yet some other embodiments of the present disclosure, the nucleotide sequence encoding the intracellular signaling domain comprises the nucleotide sequence encoding an intracellular signaling domain including CD28, 4-1BB and CD3ζ, more preferably, the intracellular signaling domain contains or consists of intracellular signaling domains of CD28, 4-1BB and CD3 ζ from the N terminal to the C terminal.

In certain embodiments of the present disclosure, the nucleotide sequence encoding the intracellular signaling domain of CD28 in the CAR comprises a sequence of SEQ ID NO: 9.

In certain embodiments of the present disclosure, the nucleotide sequence encoding the intracellular signaling domain of 4-1BB in the CAR comprises a sequence of SEQ ID NO: 11.

In certain embodiments of the present disclosure, the nucleotide sequence encoding the intracellular signaling domain of CD3ζ in the CAR comprises a sequence of SEQ ID NO: 13.

In certain embodiments of the present disclosure, the nucleotide sequence encoding the intracellular signaling domain in the CAR comprises a nucleotide sequence encoding intracellular signaling domains of CD28, 4-1BB and CD3ζ from the N terminal to the C terminal. For example, said nucleotide sequence encoding the intracellular signaling domain in the CAR comprises a sequence of SEQ ID NO: 20.

In certain embodiments of the present disclosure, said isolated nucleic acid comprises a nucleotide sequence encoding a hinge region between said antigen binding domain and said transmembrane domain, and preferably the hinge region is an IgG1H. For example, the nucleotide sequence encoding a hinge region comprises a sequence of SEQ ID NO: 5.

In certain embodiments of the present disclosure, said isolated nucleic acid comprises a nucleotide sequence encoding a leader sequence upstream said NKG2D fragment, and preferably, the leader sequence is a leading peptide of CD33. For example, the nucleotide sequence encoding a leader sequence comprises a sequence of SEQ ID NO: 17.

As mentioned above, the present disclosure provides a combination of a chimeric antigen receptor (CAR) and an accessory protein, wherein said accessory protein is DAP10 or a functional fragment thereof.

In one aspect, the present disclosure provides an isolated nucleic acid encoding the chimeric antigen receptor (CAR) and the accessory protein, wherein said CAR comprises: (a) an antigen binding domain including NKG2D or a functional fragment thereof; (b) a transmembrane domain; and (c) an intracellular signaling domain. Herein said accessory protein can be DAP10 or a functional fragment thereof. In certain embodiments of the present disclosure, the nucleotide sequence encoding antigen binding domain comprises a nucleic acid sequence encoding an NKG2D functional fragment, preferably an NKG2D fragment corresponding to amino acid residues 82-216 of human NKG2D. For example, the nucleic acid sequence encoding an NKG2D functional fragment comprises a sequence of SEQ ID NO: 1, and the nucleotide sequence encoding DAP10 comprises a sequence of SEQ ID NO: 3.

The term “nucleic acid” as used herein includes “polynucleotide,” “oligonucleotide,” and “nucleic acid molecule,” and generally means a polymer of DNA or RNA, which can be single-stranded or double-stranded, synthesized or obtained (e.g., isolated and/or purified) from natural sources, which can contain natural, non-natural or altered nucleotides, and which can contain a natural, non-natural or altered internucleotide linkage, instead of the phosphodiester found between the nucleotides of an unmodified oligonucleotide. In some embodiments, the nucleic acid does not comprise any insertions, deletions, inversions, and/or substitutions. However, it may be suitable in some instances, as discussed herein, for the nucleic acid to comprise one or more insertions, deletions, inversions, and/or substitutions. In some embodiments, the nucleic acid may encode additional amino acid sequences that do not affect the function of the CAR and which may or may not be translated upon expression of the nucleic acid by a host cell.

The present disclosure further provides isolated or purified nucleotide sequence which encodes any of the CARs or functional portions or functional variants thereof. One aspect of the disclosure also provides an isolated or purified nucleic acid comprising a nucleotide sequence which is complementary to the nucleotide sequence of any of the nucleic acids described herein or a nucleotide sequence which hybridizes under stringent conditions to the nucleotide sequence of any of the nucleic acids described herein.

The nucleotide sequence which hybridizes under stringent conditions may hybridize under high stringency conditions. By “high stringency conditions”, it is meant that the nucleotide sequence specifically hybridizes to a target sequence (the nucleotide sequence of any of the nucleic acids described herein) in an amount that is detectably stronger than non-specific hybridization. High stringency conditions include conditions which would distinguish a polynucleotide with an exact complementary sequence, or one containing only a few scattered mismatches from a random sequence that happened to have a few small regions (e.g., 3-10 bases) that matched the nucleotide sequence. Such small regions of complementarity are more easily melted than a full-length complement of 14-17 or more bases, and high stringency hybridization makes them easily distinguishable. Relatively high stringency conditions would include, for example, low salt and/or high temperature conditions, such as provided by about 0.02-0.1M NaCl or the equivalent, at temperatures of about 50-70° C. Such high stringency conditions tolerate little, if any, mismatch between the nucleotide sequence and the template or target strand, and are particularly suitable for detecting expression of any of the inventive CARs. It is generally known that conditions can be rendered more stringent by the addition of increasing amounts of formamide.

The disclosure also provides a nucleic acid comprising a nucleotide sequence that is at least about 70% or more, e.g., about 80%, about 90%, about 91%, about 92%, about 93%, about 94%, about 95%, about 96%, about 97%, about 98%, or about 99% identical to any of the nucleic acids described herein.

The nucleic acids of the disclosure can be incorporated into a recombinant expression vector. In this regard, certain embodiments of the disclosure provide a recombinant expression vector comprising any of the nucleic acids of the disclosure. As used herein, the term “recombinant expression vector” means a genetically-modified oligonucleotide or polynucleotide construct that permits the expression of an mRNA, protein, polypeptide, or peptide by a host cell, when the construct comprises a nucleotide sequence encoding the mRNA, protein, polypeptide, or peptide, and the vector is contacted with the cell under conditions sufficient to have the mRNA, protein, polypeptide, or peptide expressed within the cell. The vectors of the disclosure are not naturally-occurring as a whole. However, parts of the vectors can be naturally-occurring. The inventive recombinant expression vectors can comprise any type of nucleotides, including, but not limited to DNA and RNA, which can be single-stranded or double-stranded, synthesized or obtained in part from natural sources, and which can contain natural, non-natural or altered nucleotides. The recombinant expression vectors can comprise naturally-occurring or non-naturally-occurring internucleotide linkages, or both types of linkages. Preferably, the non-naturally occurring or altered nucleotides or internucleotide linkages do not hinder the transcription or replication of the vector.

In certain embodiment, the recombinant expression vector of the disclosure can be any suitable recombinant expression vector, and can be used to transform or transfect any suitable host cell. Suitable vectors include those designed for propagation and expansion or for expression or both, such as plasmids and viruses. The vector can be selected from the group consisting of the pUC series (Fermentas Life Sciences, Glen Burnie, Md.), the pBluescript series (Stratagene, LaJolla, Calif.), the pET series (Novagen, Madison, Wis.), the pGEX series (Pharmacia Biotech, Uppsala, Sweden), and the pEX series (Clontech, Palo Alto, Calif.). Bacteriophage vectors, such as λGT10, λGTl1, λZapII (Stratagene), λEMBL4, and λNM1 149, also can be used. Examples of plant expression vectors include pBI01, pBI101.2, pBI101.3, pBI121 and pBIN19 (Clontech). Examples of animal expression vectors include pEUK-Cl, pMAM, and pMAMneo (Clontech). The recombinant expression vector may be a viral vector, e.g., a retroviral vector or a lenti viral vector.

The recombinant expression vector may include a natural or non-natural promoter, which is operably linked to the nucleotide sequence encoding the CAR (including its functional parts and functional variants) or to a nucleotide sequence that is complementary or hybridized to the nucleotide sequence encoding the CAR nucleotide sequence. The choice of promoter, such as how strong, weak, inducible, tissue-specific and development-specific, is within the abilities of those skilled in the art. Similarly, the connection of nucleotide sequence and promoter is within the ordinary skill of those skilled in the art. The promoter can be a non-viral promoter or a viral promoter, such as EF1α promoter, cytomegalovirus (CMV) promoter, SV40 promoter, and RSV promoter. The EF1α promoter is derived from the EF1α promoter from the human targeted elongation factor 1α (EF1A) gene. In certain embodiments of the present disclosure, the EF1α promoter is used in the recombinant expression vector, which has, for example, a nucleotide sequence of SEQ ID NO: 15.

In one aspect of the present disclosure, the present disclosure provides a recombinant expression vector comprising a nucleotide sequence encoding any of the CARs described above, which comprises: (a) an antigen binding domain including NKG2D or a functional fragment thereof; (b) a transmembrane domain; and (c) an intracellular signaling domain.

In one aspect, the present disclosure provides a recombinant expression vector of a chimeric antigen receptor (CAR) and an accessory protein, wherein said CAR comprises: (a) an antigen binding domain including NKG2D or a functional fragment thereof; (b) a transmembrane domain; and (c) an intracellular signaling domain. Herein said accessory protein can be DAP10 or a functional fragment thereof. In one aspect of the present disclosure, the recombinant expression vector of a chimeric antigen receptor (CAR) and an accessory protein expresses said CAR or said accessary protein in different recombinant expression vectors. In one preferable embodiment of the present disclosure, the recombinant expression vector of a chimeric antigen receptor (CAR) and an accessory protein expresses said CAR or said accessary protein in one recombinant expression vector.

In certain embodiments of the present disclosure, in the recombinant expression vector, the nucleotide sequence encoding antigen binding domain comprises a nucleic acid sequence encoding an NKG2D functional fragment, preferably an NKG2D fragment corresponding to amino acid residues 82-216 of human NKG2D. For example, the nucleic acid sequence encoding an NKG2D functional fragment comprises, consists of SEQ ID NO: 1.

In certain embodiments of the present disclosure, in the recombinant expression vector, the nucleotide sequence encoding the transmembrane domain comprises the nucleotide sequence encoding a transmembrane domain of CD8 and/or CD28. In yet certain other embodiments of the present disclosure, the nucleotide sequence encoding the transmembrane domain comprises the nucleotide sequence encoding a transmembrane domain of CD28. For example, the nucleotide sequence comprises a sequence of SEQ ID NO: 7.

In certain embodiments of the present disclosure, in the recombinant expression vector, the nucleotide sequence encoding the intracellular signaling domain comprises one or more of the nucleotide sequence encoding an intracellular signaling domain of CD28, 4-1BB or CD3ζ. In yet certain embodiments of the present disclosure, the nucleotide sequence encoding the intracellular signaling domain comprises a nucleotide sequence encoding an intracellular signaling domain including CD28, 4-1BB and CD3ζ. More preferably, the intracellular signaling domain contains or consists of intracellular signaling domains of CD28, 4-1BB and CD3ζ from the N terminal to the C terminal.

In certain embodiments of the present disclosure, in the recombinant expression vector, a nucleotide sequence encoding the intracellular signaling domain of CD28 in the CAR comprises a sequence of SEQ ID NO: 9.

In certain embodiments of the present disclosure, in the recombinant expression vector, a nucleotide sequence encoding the intracellular signaling domain of 4-1BB in the CAR comprises a sequence of SEQ ID NO: 11.

In certain embodiments of the present disclosure, in the recombinant expression vector, a nucleotide sequence encoding the intracellular signaling domain of CD3ζ in the CAR comprises a sequence of SEQ ID NO: 13.

In certain embodiments of the present disclosure, in the recombinant expression vector, the nucleotide sequence encoding intracellular signaling domain in the CAR comprises nucleotide sequence encoding intracellular signaling domains of CD28, 4-1BB and CD3ζ from the N terminal to the C terminal, for example, said the nucleotide sequence encoding intracellular signaling domain in the CAR comprises a sequence of SEQ ID NO: 20.

In certain embodiments of the present disclosure, in the recombinant expression vector, comprises a nucleotide sequence encoding a hinge region between said antigen binding domain and said transmembrane domain, preferably the hinge region is an IgG1H. For example, the nucleotide sequence encoding a hinge region comprises a sequence of SEQ ID NO: 5.

In certain embodiments of the present disclosure, in the recombinant expression vector, comprises a nucleotide sequence encoding a leader sequence upstream said NKG2D fragment, preferably, the leader sequence is a leading peptide of CD33. For example, the nucleotide sequence encoding a leader sequence comprises a sequence of SEQ ID NO: 17.

In certain embodiments of the present disclosure, the recombinant expression vector comprises a nucleic acid control element positioned upstream said leader sequence, preferably, the nucleic acid control element is a Kozak sequence. For example, the nucleotide sequence encoding a nucleic acid control element comprises a sequence of SEQ ID NO: 16.

In certain embodiments of the present disclosure, the recombinant expression vector comprises a promotor positioned upstream said leading peptide of the CAR, preferably, the promotor is an EF1α promoter, for example, the promotor comprises a nucleotide sequence of SEQ ID NO: 15.

In certain embodiments of the present disclosure, in the recombinant expression vector, the nucleotide sequence encoding DAP10 comprises a nucleotide sequence of SEQ ID NO: 3.

In certain embodiments of the present disclosure, the nucleic acid and the recombinant expression vector additionally includes a nucleic acid sequence of IRES between the nucleic acid sequence encoding said CAR and said DAP10. For example, the nucleic acid sequence of IRES comprises a sequence of SEQ ID NO: 19.

The present disclosure further provides host cells expressing said chimeric antigen receptor (CAR). The present disclosure provides host cells expressing a combination of said chimeric antigen receptor (CAR) and an accessary protein such as DAP10. The present disclosure further provides host cells containing the nucleic acid and the recombinant expression vector as mentioned above.

As used herein, the term “host cell” refers to any type of cell that can contain the inventive recombinant expression vector. The host cell can be a eukaryotic cell, e.g., plant, animal, fungi, or algae, or can be a prokaryotic cell, e.g., bacteria or protozoa. The host cell can be a cultured cell or a primary cell, i.e., isolated directly from an organism, e.g., a human. The host cell can be an adherent cell or a suspended cell, i.e., a cell that grows in suspension. Suitable host cells are known in the art and include, for instance, DH5a E. coli cells, Chinese hamster ovarian cells, monkey VERO cells, COS cells, HEK293 cells, and the like. For purposes of amplifying or replicating the recombinant expression vector, the host cell may be a prokaryotic cell, e.g., a DH5α cell. For purposes of producing a recombinant CAR, the host cell may be a mammalian cell. The host cell may be a human cell. While the host cell can be of any cell type, can originate from any type of tissue, and can be of any developmental stage, the host cell may be a peripheral blood lymphocyte (PBL) or a peripheral blood mononuclear cell (PBMC).

In certain embodiments of the present disclosure, the host cell is a T cell. For purposes herein, the T cell can be any T cell, such as a cultured T cell, e.g., a primary T cell, or a T cell from a cultured T cell line, e.g., Jurkat, SupT1, etc., or a T cell obtained from a mammal. If obtained from a mammal, the T cell can be obtained from numerous sources, including but not limited to blood, bone marrow, lymph node, the thymus, or other tissues or fluids. T cells can also be enriched for or purified. The T cell may be a human T cell. The T cell may be a T cell isolated from a human. The T cell can be any type of T cell and can be of any developmental stage, including but not limited to, CD4⁺/CD8⁺ double positive T cells, CD4⁺ helper T cells, e.g., Th₁ and Th₂ cells, CD8⁺ T cells (e.g., cytotoxic T cells), tumor infiltrating cells, memory T cells, naive T cells, and the like. The T cell may be a CD8⁺ T cell or a CD4⁺ T cell.

In certain embodiments of the present disclosure, the host cell is a natural killer (NK) cell. The term “NK cells” (also known as natural killer cells) refers to a type of lymphocytes that originate in the bone marrow and play an important role in the innate immune system. NK cells provide a rapid immune response against virus-infected cells, tumor cells or other stressed cells, even if antibodies and major histocompatibility complexes are not present on the cell surface. NK cells can be isolated or can be obtained from commercially available sources.

The term “isolated cell” generally refers to a cell that is substantially separated from other cells of the tissue. “Immune cells” include, for example, white blood cells (leukocytes), lymphocytes (T cells, B cells, natural killer (NK) cells) and bone marrow-derived cells (neutrophils) derived from hematopoietic stem cells (HSC) produced in bone marrow. Cells, eosinophils, basophils, monocytes, macrophages, dendritic cells). “T cells” include all types of immune cells that express CD3, including T helper cells (CD4⁺ cells), cytotoxic T cells (CD8⁺ cells), natural killer T cells, T regulatory cells (Treg), and γδT cells. “Cytotoxic cells” include CD8⁺ T cells, natural killer (NK) cells and neutrophils, which can mediate cytotoxic reactions.

The present disclosure further provides a pharmaceutical composition containing said CAR materials. In this regard, an embodiment of the disclosure provides a pharmaceutical composition comprising any of the CARs, functional portions, functional variants, nucleic acids, expression vectors, host cells (including populations thereof), and antibodies (including antigen binding portions thereof), and a pharmaceutically acceptable carrier. The inventive pharmaceutical compositions containing any of the inventive CAR materials can comprise more than one inventive CAR material, e.g., a CAR and a nucleic acid, or two or more different CARs. Alternatively, the pharmaceutical composition can comprise an inventive CAR material in combination with other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc. In a preferred embodiment, the pharmaceutical composition comprises the inventive host cell or populations thereof.

With respect to pharmaceutical compositions, the pharmaceutically acceptable carrier can be any of those conventionally used and is limited only by chemical-physical considerations, such as solubility and lack of reactivity with the active agent(s), and by the route of administration. The pharmaceutically acceptable carriers described herein, for example, vehicles, adjuvants, excipients, and diluents, are well-known to those skilled in the art and are readily available to the public. It is preferred that the pharmaceutically acceptable carrier be one which is chemically inert to the active agent(s) and one which has no detrimental side effects or toxicity under the conditions of use.

Methods for preparing administrable (e.g., parenterally administrable) compositions are known or apparent to those skilled in the art and are described in more detail in, for example, Remington: The Science and Practice of Pharmacy, Lippincott Williams & Wilkins; 21 st ed. (May 1, 2005).

The following formulations for oral, aerosol, parenteral (e.g., subcutaneous, intravenous, intraarterial, intramuscular, intradermal, interperitoneal, and intrathecal), and topical administration are merely exemplary and are in no way limiting. More than one route can be used to administer the inventive CAR materials, and in certain instances, a particular route can provide a more immediate and more effective response than another route.

For purposes of the inventive methods wherein host cells or populations of cells are administered, the cells can be cells that are allogeneic or autologous to the mammal. Preferably, the cells are autologous to the mammal.

The mammal as referred to herein can be any mammal. As used herein, the term “mammal” refers to any mammal, including, but not limited to, mammals of the order Rodentia, such as mice and hamsters, and mammals of the order Logomorpha, such as rabbits. The mammals may be from the order Carnivora, including Felines (cats) and Canines (dogs). The mammals may be from the order Artiodactyla, including bovines (cows) and swines (pigs) or of the order Perssodactyla, including Equines (horses). The mammals may be of the order Primates, Ceboids, or Simoids (monkeys) or of the order Anthropoids (humans and apes). Preferably, the mammal is a human.

In one aspect of the present disclosure, the pharmaceutical composition can be for use in the treatment or prevention of cancer.

The present disclosure also provides the use of the CAR, the combination of the CAR with an accessary protein, the nucleic acid or the recombinant expression vector or the host cells in treating or preventing cancer, or in manufacturing a medicament for treating or preventing cancer. The cancer can be any cancer, including leukemia, lymphoma, multiple myeloma or solid tumors. For example, leukemia can be acute lymphocytic leukemia, acute myelogenous leukemia, acute promyelocytic leukemia, acute lymphocytic leukemia, chronic myelogenous leukemia, chronic lymphocytic leukemia, monocytic leukemia or hairy cell leukemia. Lymphomas can be Hodgkin's lymphoma, non-Hodgkin's lymphoma; Burkitt's lymphoma; and small lymphocytic lymphoma. Solid tumors can be bladder cancer, urothelial cell carcinoma of urethra, ureter and renal pelvis, multiple myeloma, kidney cancer, breast cancer, colon cancer, head and neck cancer, lung cancer, prostate cancer, glioblastoma, osteosarcoma, liposarcoma, soft tissue sarcoma, ovarian cancer, melanoma, liver cancer, esophageal cancer, pancreatic cancer and gastric cancer. In one aspect of the present disclosure, the cancer is NKG2D-related cancer. In said cancers, the NKG2D receptor-NKG2D ligand system is expressed in cancer cells and plays a physiological and biochemical role. In cells of said cancer, NKG2D ligands are usually expressed, including UL16-binding protein (UL16-binding protein, ULBP) or MHC class I-chain-related protein AB (MHC class I-chain-related protein, MICA/B).

In certain embodiments of the present disclosure, said cancer is liver cancer.

In certain embodiments of the present disclosure, said cancer is lung cancer.

In certain embodiments of the present disclosure, said cancer is leukemia.

In certain embodiments of the present disclosure, said cancer is myeloma.

### BRIEF DESCRIPTION OF THE DRAWINGS

FIG. 1 shows flow cytometry analysis result of detecting the expression of NKG2D ligand on Raji cell line.

FIG. 2 shows construction diagram of expression vectors. A: pCCL-DRCAR-IRES-DAP10 expression vector; B: pHAGE-DRCAR expression vector.

FIG. 3 shows the nucleotide sequence and description of the insert fragment of the expression vector pCCL-DRCAR-IRES-DAP10.

FIG. 4 shows the nucleotide sequence and description diagram of the insertion fragment of the expression vector pHAGE-DRCAR.

FIG. 5 shows flow cytometry analysis results of plasmid co-transfection 293T cells expressing DRCAR. The left is the control (only with the lentivirus packaging plasmid but without the recombinant plasmid). In the middle is the lentivirus with the pHAGE-DRCAR recombinant plasmid. On the right is the lentivirus with pCCL-DRCAR-IRES-DAP10 recombinant plasmid.

FIG. 6 shows titration of lentivirus expressing DRCAR. (A) is flow cytometry analysis results of lentiviruses expressing DRCAR in different amounts. (B) is the corresponding histogram. (C) Calculate the lentivirus titer formula and result based on the analysis data of the flow cytometer.

FIG. 7 shows the effect of DR-CAR-T cells in killing cancer cells. FIG. 7 (a)-FIG. 7 (u) show the DR-CAR-T cells killing effect on 21 kinds of cancer cell line expressing NKG2D ligand. FIG. 7(v) shows the DR-CAR-T cells killing effect on Raji cells that do not express NKG2D ligand. The percentage of cancer cells death was analyzed by flow cytometry.

FIG. 8 shows DR-CAR T cells inhibit tumors in human liver cancer transplantation animal models. FIG. 8 (a) is the experimental design diagram. FIG. 8(b) is a graph showing the survival curves of liver cancer transplanted mice in the experimental group injected with 4×10⁶ T cells or DR-CAR T cells. FIG. 8(c) is a graph showing the survival curves of liver cancer transplanted mice in the experimental group injected with 5×10⁶ T cells or DR-CAR T cells.

FIG. 9 shows the survival curves of animals injected with DR-CAR T cells in lung cancer transplanted mice.

### DETAILED DESCRIPTION OF THE PREFERRED EMBODIMENTS

The inventions provided in the above section of the disclosure are further described with reference to the following examples, which are intended to illustrate the disclosure and not to limit the scope of the disclosure.

### Example 1: Cell Production, Process, and Methods

Buffy coat was obtained from the Hong Kong Red Cross Blood Transfusion Service. Peripheral blood mononuclear cells (PBMCs) were isolated from buffy coat by using Ficoll-Paque PLUS (GE Healthcare). T cells were isolated from PBMCs by using CD3/CD28 Dynabeads (Thermo). T cells isolated from PBMCs were cultured in initiate medium consisting of AIM-V medium (Thermo) supplemented with 5% human serum (Sigma), 2 mM L-glutamine (Thermo) and 50 U/ml IL-2 (Peprotech), or expansion medium consisting of AIM-V medium supplemented with 5% human serum, 2 mM L-glutamine and 300 U/ml IL-2.

All the cell lines mentioned below were obtained from ATCC, ECACC or Chinese Academy of Sciences Cell Bank.

293T cells (ATCC #CRL-3216) were cultured in DMEM medium (Thermo) supplemented with 10% FBS (Thermo), 100 U/ml penicillin (Thermo) and 100 ug/ml streptomycin (Thermo).

Chronic myelogenous leukemia cell line-K562(ATCC #CCL-243), was cultured in IMEM medium (Thermo) supplemented with 10% FBS, 100 U/ml penicillin and 100 ug/ml streptomycin.

Acute lymphocytic leukemia cell line MOLT-4 (ATCC #CRL-1582), non-Hodgkin's lymph tumor cell line KARPAS299 (ECACC #06072604), myeloma cell line-RPMI8226 (ATCC #CRM-CCL-155), NCI-H929 (ATCC #CRL-9608) and U266B1 (ATCC #TIB-196), acute T cells Leukemia cell line Jurkat (ATCC #TIB-152), gastric cancer cell line HGC27 (ECACC #94042256), lung cancer cell line NCI-H522 (ATCC #CRL-5810), breast cancer cell line MDA-MB-4355 (ATCC #HTB-129) and MDA-MB-231 (ATCC #HTB-26), bladder cancer cell line 5637 (ATCC #HTB-9), liver cancer cell line QGY7703, SMMC7721 and BEL7402, were cultured in RPMI1640 medium (Thermo) supplemented with 10% FBS, 100 U/ml penicillin and 100 U/ml streptomycin.

Cervical cancer cell line Hela (ATCC #CCL-2), neuroblastoma cell line SK-N-SH (ATCC #HTB-11) were cultured in MEM medium (Thermo) supplemented with 10% FBS, 100 U/ml penicillin and 100 U/ml streptomycin.

Lung cancer cell line A549 (ATCC #CCL-185) and prostate cancer cell line PC3 (ATCC #CRL-1435) were cultured in F12 medium (Thermo) supplemented with 10% FBS, 100 U/ml penicillin and 100 U/ml streptomycin.

Lung cancer cell line NCI-H1155 (ATCC #CRL-5818) and adenocarcinoma cell line NCI-H1355 (ATCC #CRL-5865) were cultured in serum-free ACL-4 medium (Thermo).

Construction of Retroviral Plasmids.

Lentivirus Packaging, Concentration and Purification

Lentivirus was produced by co-transfection of 3rd generation lentiviral plasmid-pMDLg/pRRE, pMD2.G, pRSV-Rev, and transfer plasmids in a ratio of 2:1:1:4 into 293T cells by calcium phosphate transfection method. The freshly collected or thawed supernatant containing lentivirus was put in a centrifuge at 300 g for 3 min to exclude cell debris in the supernatant. The supernatant was filtered by a 0.45-μm Minisart syringe filter attached to a 30-ml syringe (TERUMO). The supernatant was centrifuged at 20000 g, 4° C. for 90 minutes. After ultracentrifugation, the supernatant was removed. 1/10 of the initiate lentivirus volume of AIM-V medium was added into centrifuge tubes to re-suspend the pellets. The lentivirus suspension was mixed by pipetting. The concentrated lentivirus was aliquoted and stored in −80° C. freezer.

Titration of Lentivirus

1×10⁵ Jurkat cells were seeded into each well of 12-well plate in 1 ml RPMI1640 medium supplemented with 10% FBS, 100 U/ml penicillin and 100 ug/ml streptomycin. After overnight culture, different amounts (5 ul to 100 ul) of concentrated lentivirus were added into separate wells. The samples were duplicated or triplicated to increase accuracy. Polybrene (Sigma) was added to a final concentration of 6 ug/ml in each well. 24 hours later, cells were collected by centrifuge and re-suspended in 1 ml fresh RPMI1640 medium supplemented with 10% FBS, 100 U/ml penicillin and 100 ug/ml streptomycin. Another 48 hours later, cells were harvested and the percentage of Jurkat cells expressing CAR was determined by flow cytometry. The titer of lentivirus was calculated by the following formula.

$$Lentivirus Titer ( TU \text{/} ml ) = \frac{cell number \times \% of reporter positive cells \times dillution factor}{vector volume ( ml ) \times 100}$$

T Cells Isolation, Transduction and Cultivation

1×10⁷ PBMC were isolated for CD3⁺ cells by using Dynabeads coated with antibodies to CD3 and CD28 at a bead-to-cell ratio of 3:1. The cell and bead mixture was incubated at room temperature for 1 hour on a shaker. The CD3⁺ cells enrichment was performed by magnet and re-suspended in initiation medium at 1×10⁶ cells/ml. 24 hours later, cells were collected by centrifugation (300×g, 3 minutes). The supernatant was discarded. 5×10⁸ TU lentivirus in 500 ul AIM-V medium was added into cells and centrifuged at 2000×g for 2 hours. Cells were re-suspended in the lentivirus culture and 1.5 ml initiation was added. The cells were put back into 6-well plates and placed into an incubator (37 C, 5% CO₂). 24 hours later, Transduction was performed again. Another 24 hours later, cells were collected by centrifuge (300×g, 3 minutes) and re-suspended in 2 ml of expansion medium. The cells were put back into 6-wells plate and placed into incubator (37 degree, 5% CO₂). 72 hours later, cells were transferred to a 100-cm dish and re-suspended in expansion media at concentration of 4×10⁵ cells/ml. Transduction rate can be determined by using Flow cytometer and cytotoxicity assay can be performed when T cells are sufficiently transduced.

Flow Cytometry Analysis

To detect CAR expression on the cell surface (both T cells and Jurkat cells), 1×10⁶ cells were re-suspended in 1 ml PBS buffer and stained with biotin goat anti-human IgG (H+L) (Jason Lab) followed by streptavidin-PE (eBioscience).

Cytotoxicity Assay

Target cells were collected by centrifuge and re-suspended in PBS at a concentration of 1×10⁶ cells/ml. 5 ml of the cells were stained with 2.5 ul of Oregon Green 488 (Thermo) for 20 minutes at 37 C. 20 ml of culture medium was added to absorb the excess dye. Target cells were re-suspended in culture medium at concentration of 4×10⁵ cells/ml.

T cells were collected by centrifuge and re-suspended with the medium required by the target cells at a concentration of 1.6×10⁷ cells/ml.

T cells and target cells were mixed in a ratio of 5, 10, 20 and 40 T-cells per target cell.

| | | | | | |
|---|---|---|---|---|---|
| E:T ratio ^{Note} | 40:1 | 20:1 | 10:1 | 5:1 | 0:1 |
| target cell | 500 μL | 500 μL | 500 μL | 500 μL | 500 μL |
| T cell | 500 μL | 250 μL | 125 μL | 62.5 μL | 0 μL |
| Target cell | 0 μL | 250 μL | 375 μL | 437.5 μL | 500 μL |
| medium | | | | | |

Note:
E:T ratio = T cells and target cells ratio

Cells were incubated using an incubator for 5 hours or 8 hours accordingly. Cells were collected by centrifuge and re-suspended in 500 ul 7-AAD solution (1 ug/ml). Cells were incubated on ice for 30 minutes. Death rate was analyzed by flow cytometer.

### Example 2: Expression of NKG2D Ligands in Various Cancer Cell Lines

Detect the expression of NKG2D ligand (human MICA/B and human ULBP1-ULBP6) on different cancer cell lines to determine whether CAR with NKG2D as the antigen domain can be used to kill these cell lines.

To detect human MICA/B, resuspend 1×10⁶ cells to be tested in 0.5 ml PBS buffer, and used monoclonal mouse anti-human MICA/B (R&D Cat #MAB13001) followed by biotin goat anti-mouse IgG (H+L) and then use streptavidin-APC staining.

In order to detect human ULBP2/5/6, resuspend 1×106 cells to be tested in 0.5 ml PBS buffer, and used monoclonal mouse anti-human ULBP2/5/6 (R&D Cat #MAB1298) followed by biotin Goat anti-mouse IgG (H+L) and then used streptavidin-APC staining.

To detect human ULBP1, 1×10⁶ cells to be tested were resuspended in 0.5 ml PBS buffer, and used monoclonal mouse anti-human ULBP1 (R&D Cat #MAB1380) followed by biotin goat anti-mouse IgG (H+L) and then used streptavidin-APC staining.

To detect human ULBP3, 1×10⁶ cells to be tested were resuspended in 0.5 ml PBS buffer, and used monoclonal mouse anti-human ULBP3 (R&D Cat #MAB1517) followed by biotin goat anti-mouse IgG (H+L) and then used streptavidin-APC staining.

In order to detect human ULBP4, resuspend 1×10⁶ cells to be tested in 0.5 ml PBS buffer, and use monoclonal mouse anti-human ULBP4 (R&D Cat #AF6285) followed by biotin bovine anti-goat IgG (H+L) and then used streptavidin-APC staining.

The following 21 cancer cell lines were tested for expression of NKG2D ligands (human MICA/B and human ULBP1-ULBP6):

Chronic myelogenous leukemia cell line K562 (ATCC #CCL-243), acute lymphocytic leukemia cell line MOLT-4 (ATCC #CRL-1582), non-Hodgkin lymphoma cell line KARPAS299 (ECACC #06072604), Myeloma cell line RPMI8226 (ATCC #CRM-CCL-155), NCI-H929 (ATCC #CRL-9608) and U266B1 (ATCC #TIB-196), acute T-cell leukemia cell line Jurkat (ATCC #TIB-152), gastric cancer cell line HGC27 (ECACC #94042256), lung cancer cell line NCI-H522 (ATCC #CRL-5810), breast cancer cell line MDA-MB-435S (ATCC #HTB-129) and MDA-MB-231 (ATCC #HTB-26), bladder cancer cell line 5637 (ATCC #HTB-9), liver cancer cell line QGY7703, SMMC7721 and BEL7402, cervical cancer cell line Hela (ATCC #CCL-2), neuroblast Tumor cell line SK-N-SH (ATCC #HTB-11), lung cancer cell line A549 (ATCC #CCL-185), prostate cancer cell line PC3 (ATCC #CRL-1435), lung cancer cell line NCI-H1155 (ATCC #CRL-5818) and adenocarcinoma cell line NCI-H1355 (ATCC #CRL-5865).

Experimental results proved that NKG2D ligands (human MICA/B or human ULBP1-ULBP6) are expressed in the 21 cancer cell lines mentioned above. The 21 cancer cell lines mentioned above were tested as target cells in the following CAR T cell killing experiment.

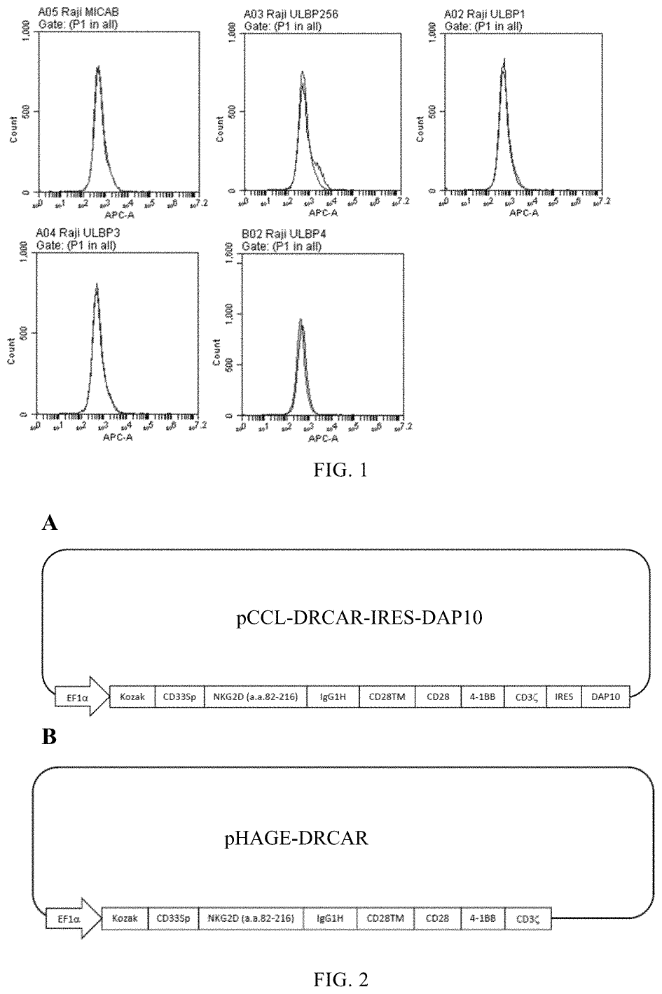

In addition, as shown in FIG. 1, Raji cells do not express NKG2D ligands (human MICA/B and human ULBP1-ULBP6). Raji cells were used as a negative control in CAR T cell killing experiments.

### Example 3: Construction of Lentiviral Vector Expressing DRCAR

1. Construction of pCCL-DRCAR-IRES-DAP10

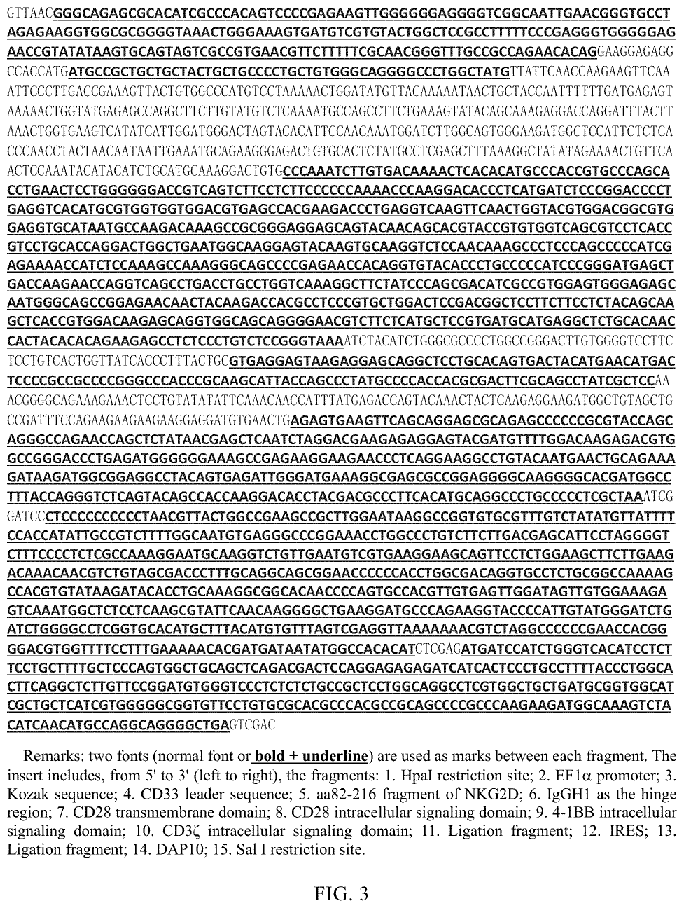

pCCL-DRCAR-IRES-DAP10 has the structure shown in FIG. 2A. pCCL-DRCAR-IRES-DAP10 was constructed by the following method.

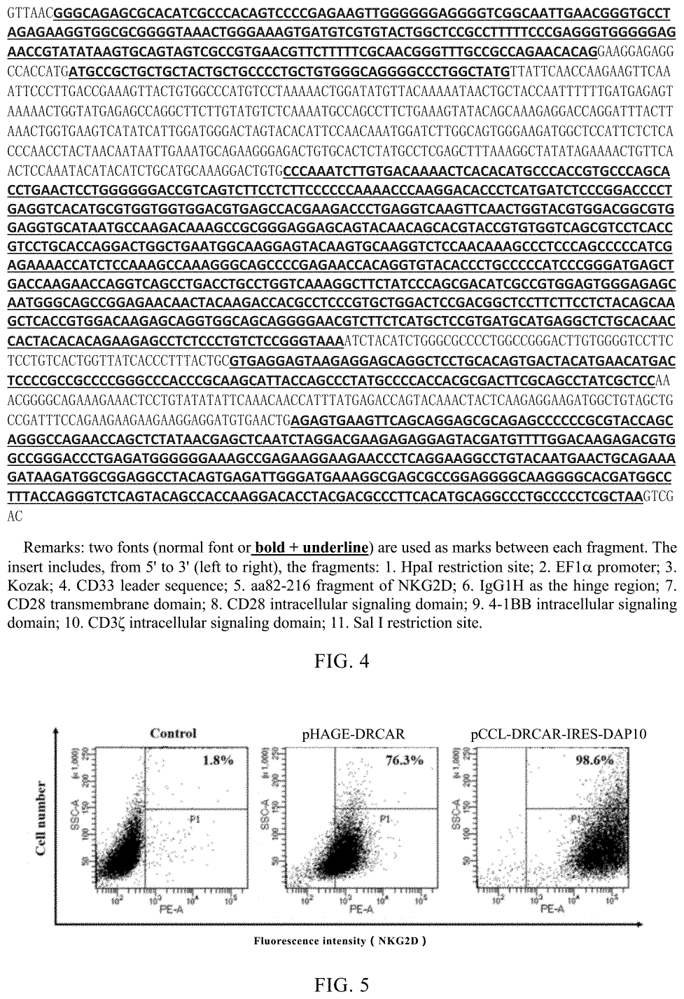

Synthesized a nucleic acid insert encoding DRCAR and DAP10, which has a nucleotide sequence as shown in FIG. 3. The insert includes, from 5′ to 3′, the fragments: 1. HpaI restriction site; 2. EF1α promoter; 3. Kozak sequence; 4. CD33 leader sequence; 5. aa82-216 fragment of NKG2D; 6. IgGH1 as the hinge region; 7. CD28 transmembrane domain; 8. CD28 intracellular signaling domain; 9. 4-1BB intracellular signaling domain; 10. CD3ζ intracellular signaling domain; 11. Ligation fragment; 12. IRES; 13. Ligation fragment; 14. DAP10; 15. Sal I restriction site.

The above insert fragment and plasmid Pax5 (Addgene, plasmid #35003) were double digested with restriction enzymes HpaI and Sal I. After the digested product was cut and recovered, it was ligated with T4 ligase overnight at 16° C. After ligation, transform the ligation product into competent E. coli, spread onto the plate, a single colony was selected and plasmid DNA was extracted for double enzyme digestion. The inserted sequence was underwent DNA sequencing. pCCL-DRCAR-IRES-DAP10 was then obtained.

2. Construction of pHAGE-DRCAR

pHAGE-DRCAR has the structure shown in FIG. 2B. The pHAGE-DRCAR was constructed by the following method.

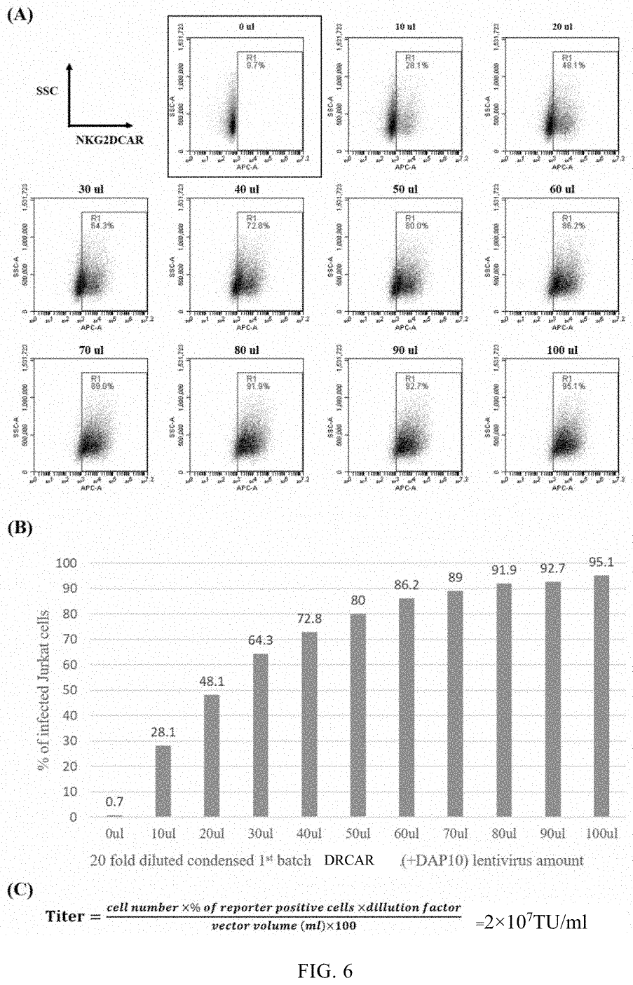

Synthesized a nucleic acid insert encoding DRCAR having a nucleotide sequence as shown in FIG. 4. The insert includes, from 5′ to 3′, the fragments: 1. HpaI restriction site; 2. EF1α promoter; 3. Kozak sequence; 4. CD33 leader sequence; 5. aa82-216 fragment of NKG2D; 6. IgG1H as the hinge region; 7. CD28 transmembrane domain; 8. CD28 intracellular signaling domain; 9. 4-1BB intracellular signaling domain; 10. CD3ζ intracellular signaling domain; 11. Sal I restriction site.

The inserted fragment and plasmid Pax5 were double-enzyme digested with restriction enzymes HpaI and Sal I. After the digested product was cut and recovered, it was ligated with the insert by using T4 ligase overnight at 16° C. After ligation, the ligated product was transformed into E. coli, spread onto the plate, and single colony was selected the next day for plasmid extraction. The plasmids underwent double enzyme digestion and sequencing. pHAGE-DRCAR was then obtained.

3. Production of Lentiviral Vectors

Using PHAGE-DRCAR or pCCL-DRCAR-IRES-DAP10 as expression plasmids, and third-generation lentiviral plasmids pMDLg/pRRE, pMD2.G and pRSV-Rev were co-transfected to prepare corresponding lentiviral vectors.

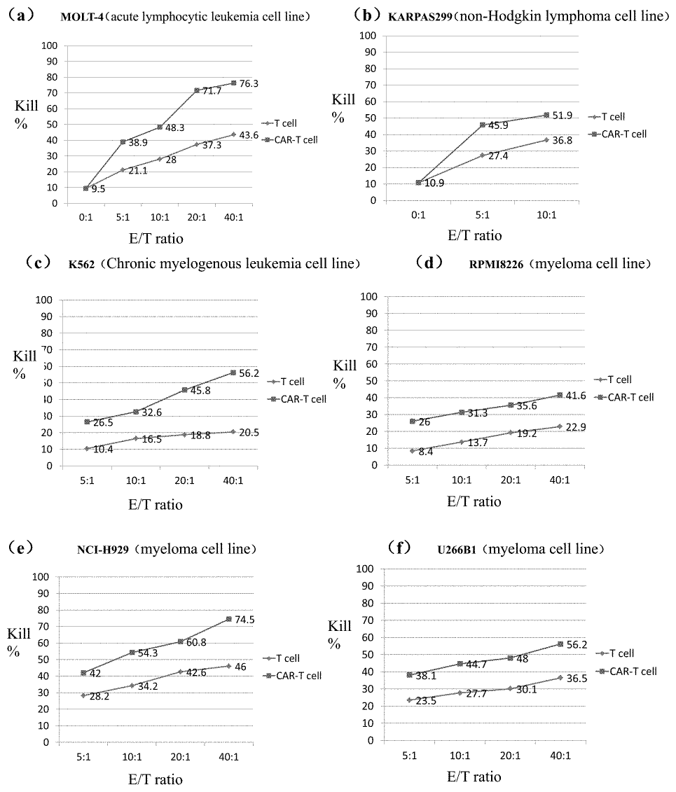

Using method recited in Example 1, expression of DRCAR by the lentivirus were measured. FIG. 5 is a flow cytometric analysis result of 293T cells co-transfected with the plasmids expressing DRCAR. The results showed that, in comparison with pHAGE-DRCAR, the expression level of DRCAR by the lentivirus containing pCCL-DRCAR-IRES-DAP10 was significantly higher.

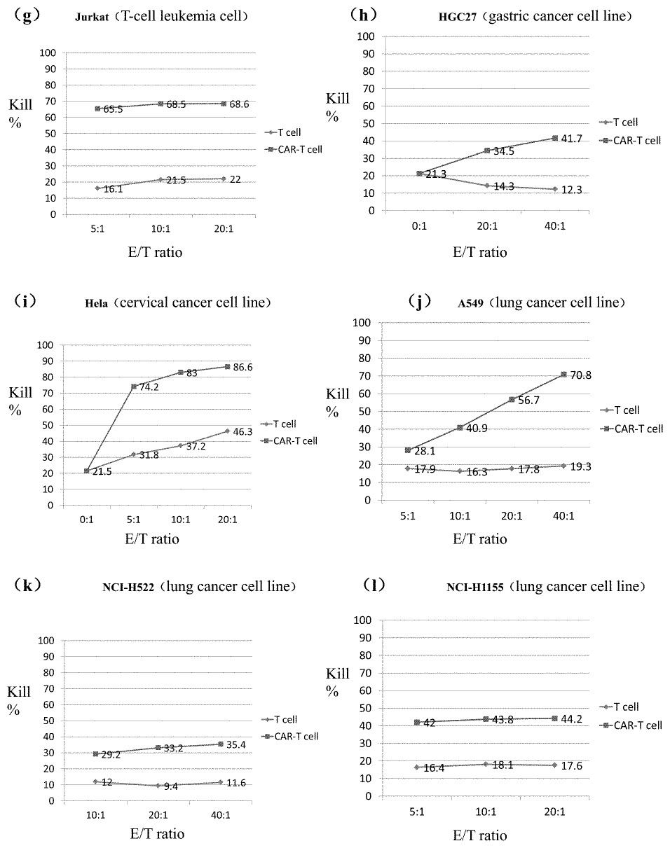

The titer of the lentivirus containing pCCL-DRCAR-IRES-DAP10 was calculated according to the method described in Example 1. FIG. 6 shows that the titer of pCCL-DRCAR-IRES-DAP10 packaged lentivirus is about 2×10⁷ TU/ml. Compared with pHAGE-DRCAR, the lentivirus containing pCCL-DRCAR-IRES-DAP10 gives a higher lentivirus titer.

### Example 4: DR-CAR-T Cells Kill Cancer Cells In Vitro

According to the method described in Example 1, T cells were isolated from human PBMC. Then, the Lentivirus containing pCCL-DRCAR-IRES-DAP10 prepared in Example 3 was used to transduce T cells.

The cytotoxicity assay was performed according to the method described in Example 1, and the specific cytotoxic effects of T cells expressing DR-CAR on various tumor cells were examined. Among them, T cells expressing DR-CAR and untransduced T cells were used as effector cells, 21 different types of cancer cell lines were used as target cells.

The Raji cell line that does not express NKG2D ligand was used as a negative control.

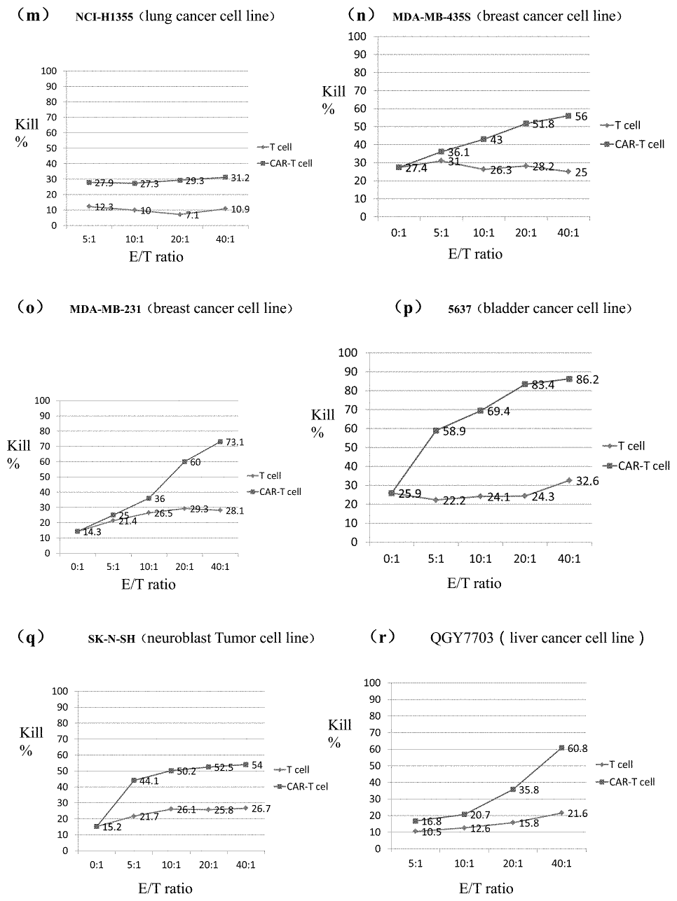

The results are shown in FIG. 7(a)-FIG. 7(u). Under the same experimental conditions, compared with normal T cells, DR-CAR-T cells significantly kill NKG2D ligand expressing target tumor cells. The tumor cells include: Chronic myelogenous leukemia cell line-K562 (ATCC #CCL-243), acute lymphocytic leukemia cell line-MOLT-4 (ATCC #CRL-1582), non-Hodgkin lymphoma cell line-KARPAS299 (ECACC #06072604), Myeloma cell line-RPMI8226 (ATCC #CRM-CCL-155), NCI-H929 (ATCC #CRL-9608) and U266B1 (ATCC #TIB-196), acute T-cell leukemia cell line-Jurkat (ATCC #TIB-152)), gastric cancer cell line-HGC27 (ECACC #94042256), lung cancer cell line-NCI-H522 (ATCC #CRL-5810), breast cancer cell line-MDA-MB-435S (ATCC #HTB-129) and MDA-MB-231 (ATCC #HTB-26), bladder cancer cell line −5637 (ATCC #HTB-9), liver cancer cell line-QGY7703, SMMC7721 and BEL7402, cervical cancer cell line-Hela (ATCC #CCL-2), neuroblast Tumor cell line-SK-N-SH (ATCC #HTB-11), lung cancer cell line-A549 (ATCC #CCL-185), prostate cancer cell line-PC3 (ATCC #CRL-1435), lung cancer cell line-NCI-H1155 (ATCC #CRL-5818) and adenocarcinoma cell line-NCI-H1355 (ATCC #CRL-5865).

As shown in FIG. 7(v), the NKG2D ligand negative cell line, Raji, the cytotoxic effect of DR-CAR-T cells did not show any difference from normal T cells.

### Example 5: DR-CAR T Cells Inhibit Tumors Growth in Human Liver Cancer Transplantation Animal Models

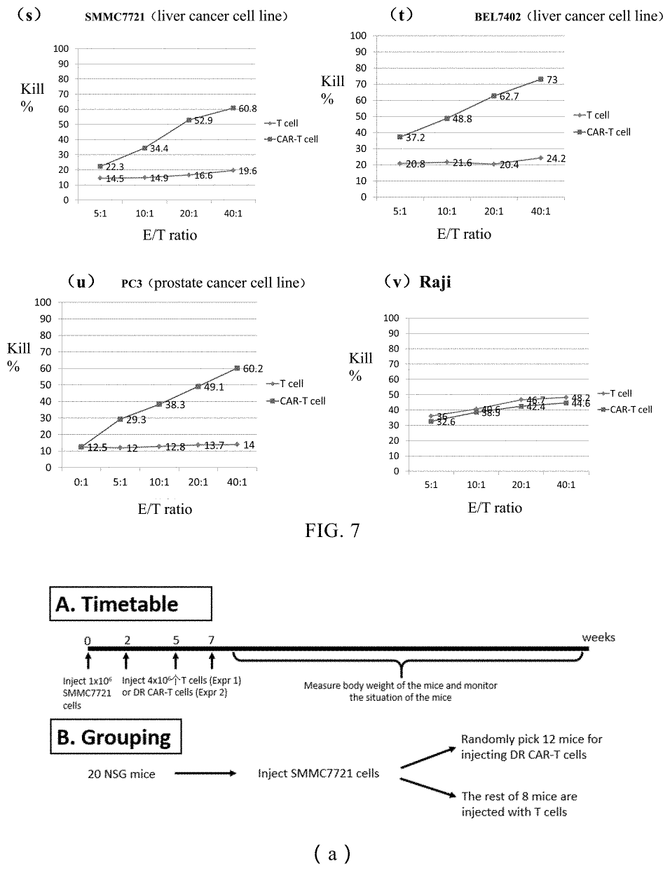

FIG. 8 (a) is the experimental design diagram.

A total of 20 experimental mice (NSG mice, 6-8 weeks old, provided by the Animal and Plant Car Facility of the Hong Kong University of Science and Technology) were divided into a DR-CAR T cell treatment group (12 randomly selected) and a T cells control group (8). All mice were intraperitoneal injected with liver cancer cells SMMC7721 (1×10⁶ cells) on day. Each group was injected with the same number of DR-CAR T cells (treatment group) or T cells (control group) in three times on the second, fifth and seventh weeks. Body weight and survival of mice was observed and recorded. The mice at the end of the experiment were characterized by death, or weight loss of 20% or more, sunken eyes or closed eyelids, unresponsive or outlier.

### Experiment 1

In the second, fifth and seventh weeks of the experiment, 4×10⁶ DR-CAR T cells (treatment group) or T cells (control group) were injected three times

The experimental results are shown in FIG. 8(b).

### Experiment 2

In the second, fifth and seventh weeks of the experiment, 5×10⁶ DRCAR T cells (treatment group) or T cells (control group) were injected three times

The experimental results are shown in FIG. 8(c).

From the experimental results, compared with the control group by using T cells that do not express DR-CAR, the DR-CAR T cells of the present disclosure can effectively protect the cancerous animals.

### Example 6: DR-CAR T Cells Inhibit Tumors in Human Lung Cancer Transplantation Animal Models

The experimental arrangement is similar to Example 5, 24 experimental mice (NSG mice, 6-8 weeks old, provided by the Animal Feeding Room of the Department of Immunology of Peking University) which 16 mice are randomly divided into DR-CAR T cell therapy group (treatment group 1 and treatment group 2) and 8 mice are in control normal T cell group. All mice were injected with 1×10⁶ lung cancer cell line, A549, on the day 0, and each group was injected DRCAR T cells (treatment group) or T cells (control group) were injected three times at 2^{nd} week (14th day), 4th week (28^{th} day) and 6^{th} week (42^{nd} day). Body weight and survival of mice was observed and recorded. The mice at the end of the experiment were characterized by death, or weight loss of 20% or more, sunken eyes or closed eyelids, unresponsive or outlier.

Treatment group 1: In the second, fourth and sixth week of the experiment, 2.5×10⁶ DRCAR T cells were injected three times

Treatment group 2: In the second, fourth and sixth week of the experiment, 5×10⁶ DRCAR T cells were injected three times

Control group: 5×10⁶ T cells were injected three times in the second, fourth and sixth week of the experiment

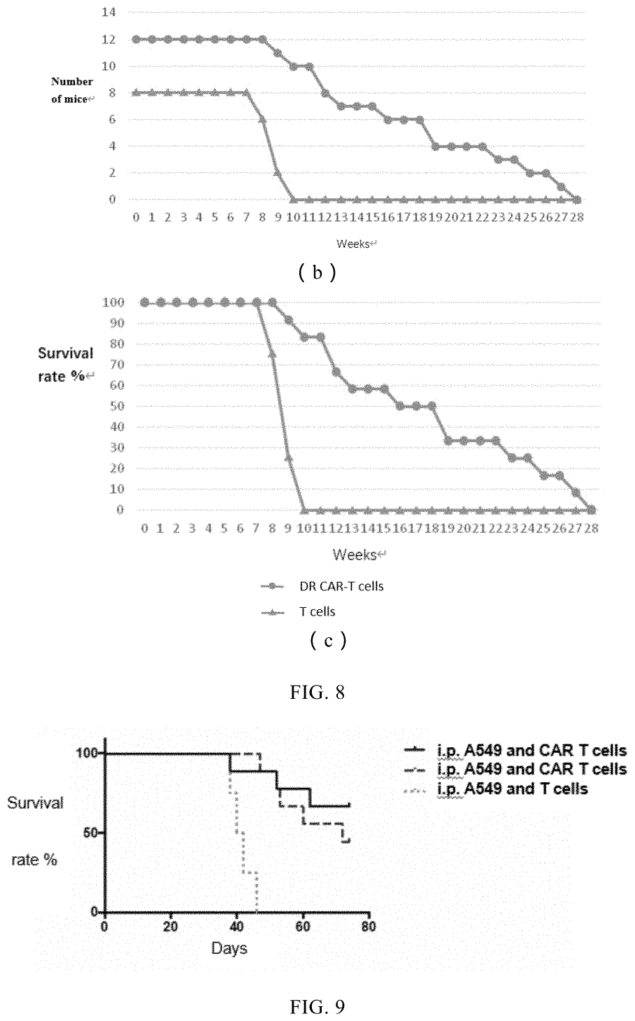

The experimental results are shown in FIG. 9.

From the experimental results, compared with the control group by using T cells that do not express DR-CAR, the DR-CAR T cells of the present disclosure can effectively protect the cancerous animals.

The above experimental results show that the DRCAR and DRCAR-T cells with the NKG2D antigen receptor structure provided by the present disclosure can effectively recognize cancer cells with NKG2D ligands and activate tumor cell-specific anti-tumor cell immune responses and kill related tumor cells. The experiments results have also proved that the DRCAR and DRCAR-T cells provided by the present disclosure can kill various cancer cells in a broad spectrum of cancers, and have proven their effectiveness in inhibiting various cancers in animal.

Unless otherwise indicated, the practice of the present disclosure will employ common technologies of organic chemistry, polymer chemistry, biotechnology, and the like. It is apparently that in addition to the above description and examples than as specifically described, the present disclosure can also be achieved in other ways. Other aspects within the scope of the disclosure and improvement of the present disclosure will be apparent to the ordinary skilled in the art. According to the teachings of the present disclosure, many modifications and variations are possible, and therefore it is within the scope of the present disclosure.

Unless otherwise indicated herein, the temperature unit “degrees” refers to Celsius degrees, namely ° C.

1. An isolated nucleic acid, comprising a nucleotide sequence encoding a chimeric antigen receptor (CAR) and a nucleic acid sequence encoding DAP10 or a functional fragment thereof, wherein the CAR comprises: (a) an antigen binding domain comprising an NKG2D fragment corresponding to amino acid residues 82-216 of human NKG2D; (b) a transmembrane domain; and (c) an intracellular signaling domain.

2. The nucleic acid of claim 1, wherein the CAR comprises a nucleic acid sequence encoding a transmembrane domain of CD8 and/or CD28.

3. (canceled)

4. The nucleic acid of claim 1, wherein the intracellular signaling domain comprises CD28, 4-1BB and CD3ζ in an order from an N terminal to a C terminal thereof.

5. The nucleic acid of claim 1, wherein the CAR further comprises a hinge region between said antigen binding domain and said transmembrane domain.

6. The nucleic acid of claim 1, wherein the CAR further comprises a leader sequence upstream said NKG2D fragment.

7. (canceled)

8. The nucleic acid of claim 1, further comprising a nucleic acid sequence of IRES between the nucleotide sequence encoding said CAR and the nucleic acid sequence encoding said DAP10 or the functional fragment thereof.

9-13. (canceled)

14. A host cell, comprising the nucleic acid of claim 1.

15. The host cell of claim 14, wherein the host cell is a T cell.

16. The host cell of claim 14, wherein the host cell is a subject to be treated.

17-22. (canceled)

23. A method for treating or preventing cancer, using the nucleic acid of claim 1.

24. The nucleic acid of claim 1, wherein the nucleic acid is part of a recombinant expression vector expressing said nucleotide sequence encoding a chimeric antigen receptor (CAR) and the nucleic acid sequence encoding DAP10 or a functional fragment thereof.

25. The nucleic acid of claim 24, wherein the recombinant expression vector comprises a nucleic acid sequence encoding a leader sequence positioned upstream of said NKG2D fragment.

26. The nucleic acid of claim 25, wherein the recombinant expression vector further comprises a nucleic acid control element positioned upstream of the nucleic acid encoding said leader sequence.

27. The nucleic acid of claim 24, wherein the recombinant expression vector further comprises a promotor positioned upstream of the nucleotide sequence encoding the CAR.

28. The nucleic acid of claim 24, wherein the recombinant expression vector is a viral vector.

29. The method of claim 23, wherein said cancer is leukemia, lymphoma, multiple myeloma or a solid tumor.

30. The method of claim 29, wherein said leukemia is acute lymphocytic leukemia, acute myelogenous leukemia, acute promyelocytic leukemia, acute lymphocytic leukemia, chronic myelogenous leukemia, chronic lymphocytic leukemia, monocytic leukemia or hairy cell leukemia.

31. The method of claim 29, wherein said lymphoma is Hodgkin's lymphoma, non-Hodgkin's lymphoma, Burkitt's lymphoma, or small lymphocytic lymphoma.

32. The method of claim 29, wherein said solid tumor is bladder cancer, urothelial cell carcinoma of urethra, ureter and renal pelvis, multiple myeloma, kidney cancer, breast cancer, colon cancer, head and neck cancer, lung cancer, prostate cancer, glioblastoma, osteosarcoma, liposarcoma, soft tissue sarcoma, ovarian cancer, melanoma, liver cancer, esophageal cancer, pancreatic cancer, or gastric cancer.

33. The method of claim 29, wherein said cancer is liver cancer or lung cancer.
